(19) United States
(12) Patent Application Publication
(10) Pub. No.: US 2003/0157502 A1
(43) Pub. Date: Aug. 21, 2003

(54) Chemotherapeutant screening method

(76) Inventors: Peter Krammer, Heidelberg (DE); Soren Eichhorst, Heidelberg (DE); Min Li-Weber, Heidelberg (DE); Martina Muller-Schilling, Heidelberg (DE)

Correspondence Address: Steven J Hultquist, Intellectual Property Technology Law, PO Box 14329, Research Triangle Park, NC, 27709, US

(21) Appl. No.: 10/169,059

(22) PCT Filed: Dec. 22, 2000
(86) PCT No.: PCT/DE00/04643

(30) Foreign Application Priority Data
Dec. 23, 1999 (DE) 19962828.9

(51) Int. Cl. C12Q 1/68

(52) U.S. Cl. 435/6

(57) ABSTRACT

Disclosed is a method for determining whether a test compound is effective for a chemotherapy. The inventive method comprises the following steps: a) a DNA sequence is provided, whereby said sequence comprises site AP-1 represented in FIG. 5A as a constituent of a promoter, preferably promoter CD95L, which is functionally linked to a reporter gene or the CD95L gene, b) the DNA sequence a) is brought into contact with the test compound in a cellular assay, and c) activation of the promoter is determined, said activation indicating that the test compound is effective for a chemotherapy.

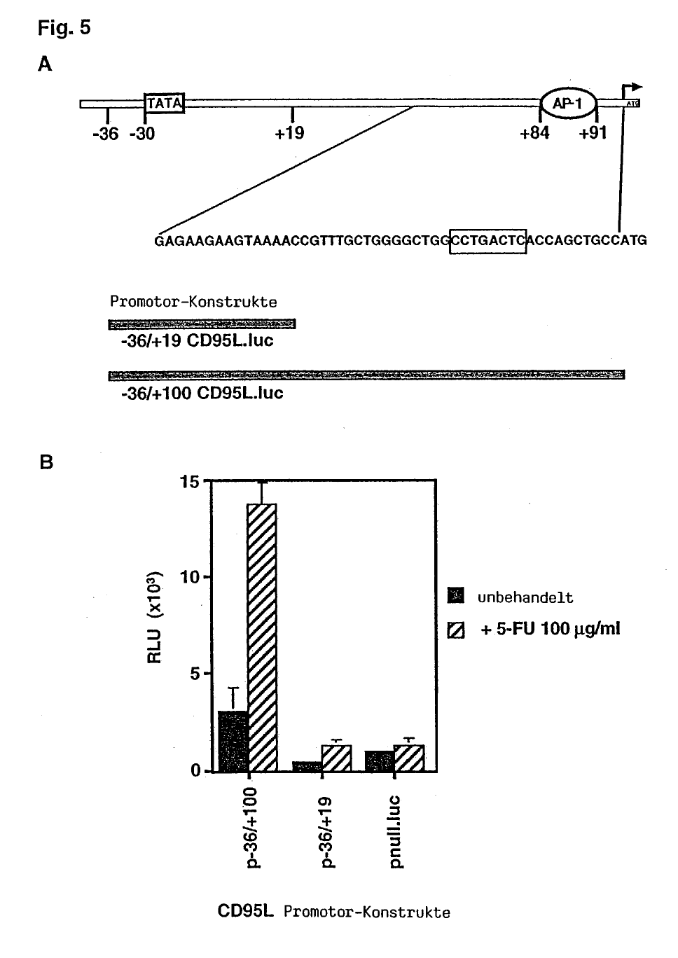

The present invention relates to a method of studying whether a test compound is effective for a chemotherapy, the method comprising the steps of: a) providing a DNA sequence comprising the AP-1 site shown in FIG. 5A as a constituent of a promoter, preferably the CD95L promoter, functionally linked with the CD95L gene or a reporter gene, b) contacting the DNA sequence from a) with the test compound in a cellular assay, and c) determining the promoter activation, said activation indicating that the test compound is effective for a chemotherapy.

The conversion of normal cells into cancer cells takes place in several substeps where mutation of cellular genes (proto-oncogenes and tumor suppressor genes) or the acquisition of viral oncogenes occurs. Cancer-relevant changes are the activation of proto-oncogenes by mutations, gene amplification, overexpression or chromosome translocations as well as the inactivation of tumor suppressor genes by mutations, e.g. deletions. As regards the prevention or treatment of cancer, the tumor suppressor genes have proved to be of special interest since they obviously offer new possibilities for the treatment of cancer. However, the previous studies show that although tumor suppressor genes represent an important objective of treating cancer, it has hardly been possible to put the results obtained into practice thus far, so that treatment of cancer has substantially been the cytostatic treatment of cancer patients till now, whether by rather non-specifically effective cytostatic agents or by radiation treatment. The cytostatic treatment of tumors by means of cytostatic agents or radiation treatment is, however, accompanied by considerable drawbacks based on serious side-effects. Therefore, there is a major requirement for identifying new chemotherapeutic agents, above all those having more specific effects or fewer side-effects.

The present invention is thus based on the technical problem of providing methods of identifying compounds effective for a chemotherapy, e.g. for controlling cancer.

This technical problem is solved by providing the embodiments characterized in the claims.

It is pointed out in the present invention that a specific sequence in the CD95L promoter, which contains an AP-1 site, is responsible for a major increase in the CD95L gene expression by chemotherapeutic agents. The increase in the transcription rate for the CD95L gene results, directly or indirectly, in an apoptosis of tumor cells. Hence new compounds useful as chemotherapeutic agents can be identified by screening methods which use the above DNA sequence as a probe.

The chemotherapeutic agents comprise inter alia compounds which can induce apoptosis in tumor cells. In this connection, it turned out that the CD95(Apo-1/Fas)/CD95L system, which plays a key function when apoptosis is regulated, is involved in the induction of apoptosis in lymphoid and non-lymphoid tissues, e.g. liver tissue and intestinal tissue. The upward regulation of the CD95 ligand (CD95L) is certainly one of the most important mechanisms by means of which anticancer agents can induce apoptosis in tumors. In the course of the studies culminating in the present invention it was found out that the upward regulation of CD95L is functionally relevant at least in the liver cells and proceeds on the transcription regulation level. It turned out that the stimulation of CD95L expression by chemotherapeutic agents (e.g. 5-fluorouracil (5-FU) and etoposide) is dependent on the basal CD95L promoter (136 bp). An AP-1 element within the 5′-non-translated region (5′UTR) downstream of a “TATA box” is required for promoter induction. This increase in the transcription rate is dependent on transcription and translation and is mediated by binding a Jun/Fos heterodimer to this AP-1 site. In summary, it may be assumed that apoptosis induced by chemotherapeutic agents in liver cells presumably proceeds according to the following mechanism: On the one hand, chemotherapeutic agents induce the CD95 gene via a transcriptionally regulated p53-dependent mechanism. On the other hand, chemotherapeutic agents influence the SAPK/JNK pathway, which ultimately results in the upward regulation of CD95L. When both molecules (CD95 and CD95L) are present on a cell surface, this cell can either commit “suicide” or kill its neighboring cell by means of a mechanism referred to as “fratricide”. It also turned out that the effect of the chemotherapeutic agents on the CD95L promoter is delayed. 20 to 25 hours following treatment with chemotherapeutic agents there is a sharp increase in the mRNA levels and the promoter activity measured by activating the luciferase gene as a reporter gene and said increase continues afterwards. Interestingly enough, various chemotherapeutic agents (e.g. 5-FU and etoposide) show a synergistic effect as regards the activation of the CD95L promoter. These observations can be used for improving the chemotherapy for different tumors, e.g. as discussed above already also for establishing a screening system to isolate new chemotherapeutic agents and contribute to a better understanding of the side-effects occurring when patients are given chemotherapeutic agents. Hence it should also be possible to isolate or develop new chemotherapeutic agents which permit the treatment of tumors already resistant to the currently known chemotherapeutic agents. It should also be possible to discover chemotherapeutic agents having increased specificity, thereby reducing the side-effects on healthy cells.

An embodiment of the present invention thus relates to a method of studying whether a test compound is effective for a chemotherapy, the method comprising the steps of:

a) providing a DNA sequence comprising the AP-1 site shown in FIG. 5A as a constituent of a promoter, preferably the CD95L promoter, functionally linked with a reporter gene or the CDS95L gene;

b) contacting the DNA sequence from a) with the test compound in a cellular assay; and

c) determining the activation of the promoter, said activation indicating that the test compound is effective for a chemotherapy.

The term “AP-1 site” used herein comprises the AP-1 site (a) having the sequence shown in FIG. 5A and (b) having the sequence differing from the sequence shown in FIG. 5A by one or more insertions, substitutions or additions of nucleotides, these modifications failing to result in a loss of the AP-1 site as regards the capability of activation by the factors discussed herein.

The test compounds may be very different compounds, both naturally occurring compounds and synthetic, organic and inorganic compounds as well as polymers (e.g. oligopeptides, polypeptides, oligonucleotides and polynucleotdies) as well as small molecules, antibodies, sugar, fatty acids, nucleotides and nucleotide analogs, analogs of naturally occurring structures (e.g. peptide “imitators”, nucleic acid analogs, etc.) and numerous other compounds.

For the production of the DNA sequence from step a), a person skilled in the art can use common in vitro recombination methods, as described e.g. in Sambrook J., E. F. Fritsch, T. Maniatis (1989). Molecular Cloning. A Laboratory Manual, 2nd edition, Cold Spring Harbor Laboratory Press, Cold Spring Harbor, N.Y.

Furthermore, the person skilled in the art can use common cells, such as HepG2 or Hep3B, for the production of the cellular assay and introduce the DNA sequence from step into them a) by means of common methods, such as transfection or electroporation. In this connection, reference is also made to Sambrook J. et al., supra. The compound can then be added to the cells via the medium, for example. Here, it should be noted that the test compound contacts the DNA sequence from step a) under conditions enabling specific binding. Thereafter, it is preferably determined in the same test system whether the transcription of the promoter functionally linked with the reporter gene was induced by the presence of the test compound. The data found as regards the transcription rate are compared preferably with the data of a second test system only differing from the first one by the absence of the test compound. Fundamentally suited assay formats for identifying compounds influencing CD95L expression are well known in the biotechnological and pharmaceutical industries and additional assays and variations of the above assay provided for the purpose of illustration are obvious to the person skilled in the art.

Along with the CD95L promoter, other promoters such as the trail-receptor promoter or the CD95 promoter are also suited for the method according to the invention. Changes as regards the promoter activity can be measured by any suitable method. Changes in the expression level can be studied using methods well known to the person skilled in the art. They comprise monitoring the mRNA concentration (e.g. using suitable probes or primers), immunoassays as regards the protein concentration (e.g. by means of the below described antibodies), RNAse protection assays, amplification assays or any other means suited for detection and known in the art. Further detection methods as regards the activation of the promoter are dependent on the specific properties of the respective reporter gene.

Compounds effective for a chemotherapy can also be screened for on a large scale, e.g. by screening a very large number of candidate compounds in substance libraries, the substance libraries being able to contain synthetic or natural molecules. Thus, the test compound of a preferred embodiment of the method according to the invention forms part of a substance library.

The above method according to the invention can be modified by means of protocols described in scientific literature and patent literature and known in the art. For example, a large number of possibly useful molecules can be screened in a single test. For example, when a field of 1000 compounds shall be screened, in principle all of the 1000 compounds can be placed in a microtitration plate well and tested at the same time. When a promoter activator is discovered, the pool of 1000 can be divided into 10 pools of 100 and the process can be repeated until an individual activator is identified. In any case, the production and simultaneous screening of large libraries from synthetic molecules can be carried out by means of well known methods of combinatorial chemistry, see e.g. van Breemen, Anal. Chem. 69 (1997), 2159-2164 and Lam, Anticancer Drug Des. 12 (1997), 145-167.

The method according to the invention can also be accelerated greatly as high throughput screening. The assays described herein as regards promoter activation can be modified correspondingly so that they can be used in such a method. It is obvious to the person skilled in the art that a number of methods are available for this purpose.

In addition, a large number of possibly useful promoter activity-modifying compounds can be screened in extracts of natural products as a starting material. Such extracts may be derived from a large number of sources, e.g. the following species: fungi, actinomycetes, algae, insects, protozoa, plants and bacteria. The extracts showing activity can then be analyzed for isolating the active molecule. See e.g. Turner, J. Ethnopharmacol. 51 (1-3) (1996), 39-43, and Suh, Anticancer Res. 15 (1995) 233-239.

In a particularly preferred embodiment of the method according to the invention the DNA sequence, in step a) which contains the AP-1 site corresponds to the nucleic acid sequence shown in FIG. 5A from positions −36 to +100 or a fragment thereof (the fragment still containing the AP-1 site), most preferably from positions +84 to +91.

In another particularly preferred embodiment of the method according to the invention the reporter gene is a CAT, luciferase, LacZ or GFP gene.

Another embodiment of the present invention relates exclusively to a medicament comprising a mixture of at least two compounds identified as defined in the method according to the invention and/or supporting the expression of the CD95L gene. Since it could be shown in the below examples that the simultaneous administration of different compounds connected with the support of CD95L expression yields synergistic effects, such a procedure is of special therapeutic interest, since e.g. the dosage of the individual substances can (extensively) be reduced and thus potential detrimental side-effects can be eliminated or at least reduced. The medicament according to the invention is preferably available in combination with a pharmaceutically compatible carrier. Suitable carriers are e.g. phosphate-buffered common salt solutions, water, emulsions, e.g. oil/water emulsions, wetting agents, sterile solutions, etc.

The medicaments according to the invention can be administered orally or preferably parenterally. The methods of parenteral administration comprise the topical, intra-arterial, intra-muscular, subcutaneous, intramedullary, intrathekal, intraventricular, intravenous, intraperitoneal or intranasal administration. The suitable dose is determined by the attending physician and depends on various factors, e.g. on the patient's age, sex and weight, the stage of the disease, e.g. the tumor, the kind of administration, etc.

The present invention finally relates to the use of the medicament according to the invention for treating different diseases, in particular cancer and autoimmune diseases, such as diabetes, multiple sclerosis and rheumatoid arthritis.

The figures show:

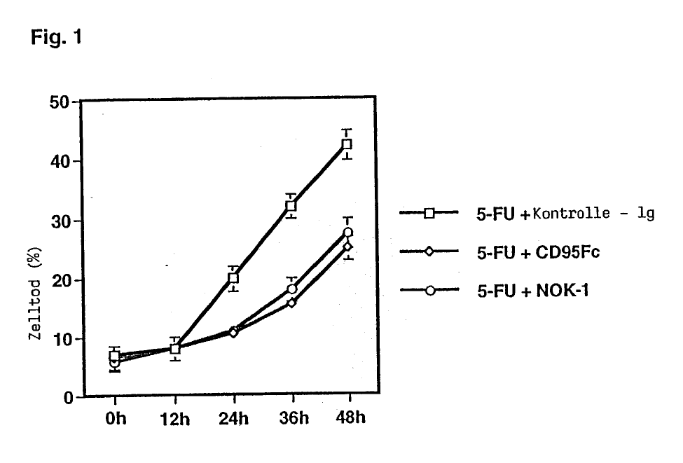

FIG. 1: Chemotherapeutic agents effect CD95-mediated death in HepG2 cells HepG2 cells were cultured almost up to confluence and then incubated with 100 μg/ml 5-FU over the indicated periods in the presence of either an isotype-adapted control antibody or the anti-CD95L antibody NOK-1 (50 μg/ml). The same experiment was carried out with the addition of CD95-Fc (50 μg/ml). Apoptosis was determined by PI exclusion and measurement of the forward/side scatter (FSC/SSC). The data shown are average values with standard deviations from three samples. Different experiments with similar results were carried out.

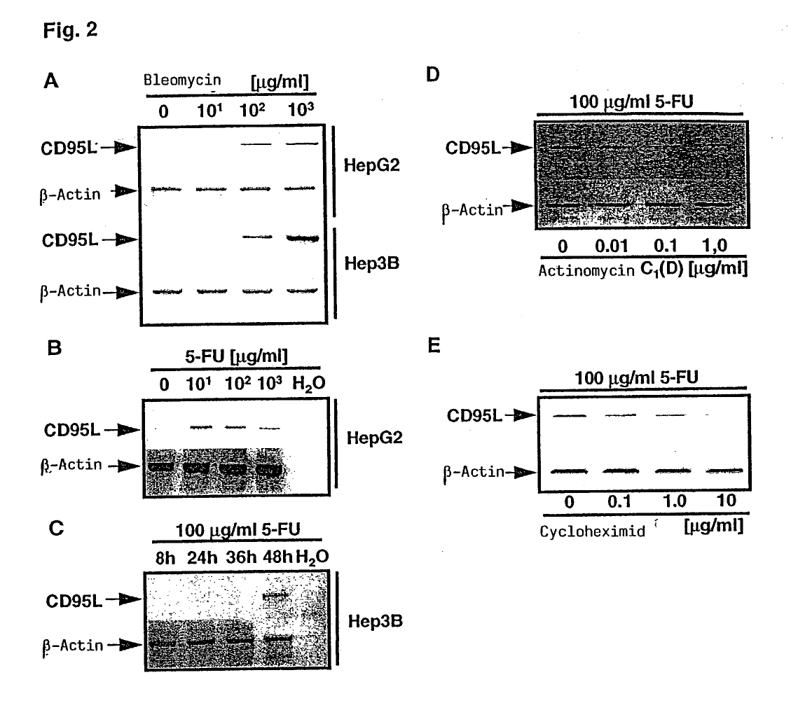

FIG. 2: Following stimulation using different chemotherapeutic agents CD95L-mRNA is induced and this induction is regulated on the transcription level PCR analyses of HepG2 and Hep3B cells were carried out. Whole RNA was extracted and RT-PCR was carried out as described in Example 1. A+B: HepG2 and Hep3B cells were incubated for 36 hours with the indicated concentrations of bleomycin or 5-FU. C: Hep3B cells were incubated with 100 μg/ml 5-FU over the indicated periods. D+E: 5-FU (100 μg/ml) was added to HepG2 cells for 48 hours and then either the transcription inhibitor actinomycin C1 (D) or the translation inhibitor cycloheximide (E) was added at given concentrations.

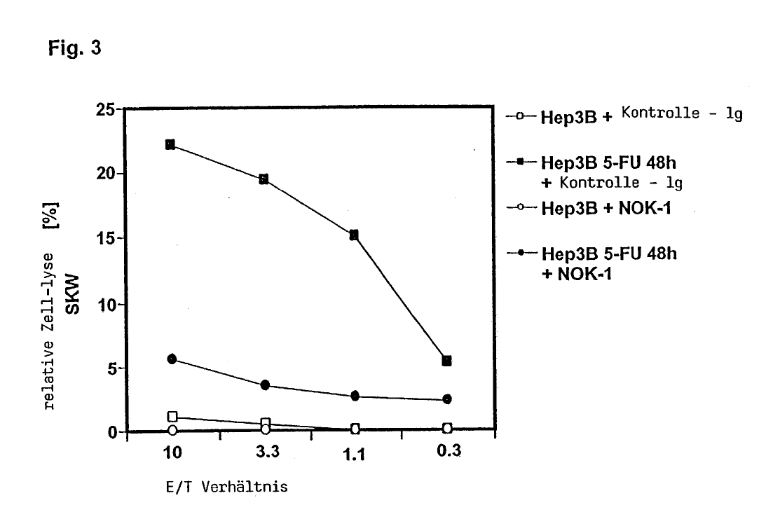

FIG. 3: The treatment with chemotherapeutic agents results in an upward regulation of functional CD95L A ⁵¹Cr release assay with Hep3B cells as effectors and SKW6.4 cells as target was used. Hep3B cells were cultured in 96-well plates treated with 5-FU. After 48 hours, the chemotherapeutic agents were removed from the culture medium and ⁵¹Cr-labeled SKW6.4 cells were added with either a control antibody or the anti-CD95L antibody NOK-1. Following incubation overnight, the supernatants were measured in a gamma counter. Relative lysis was calculated as indicated in Example 1. E/T is the ratio between effector cells and target cells. Every series of concentration was carried out threefold. The diagram represents an experiment from a series of independent experiments having the same result.

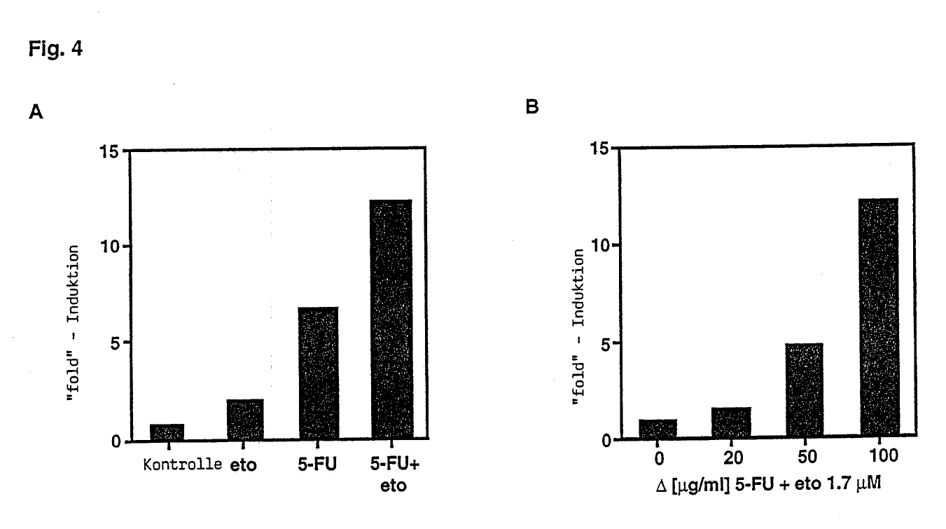

FIG. 4: The upward regulation of the CD95L promoter by chemotherapeutic agents is potentiated by simultaneous stimulation with different chemotherapeutic compounds and depends on the concentration A: Potentiating effect of different chemotherapeutic agents on the CD95L promoter. Hep3B cells were transfected with the −36/+100 construct, and the luciferase activity was measured following 48-hour treatment with 5 μM etoposide (eto), 100 μg/ml 5-FU or a combination of both preparations. Every bar represents the calculated value from treated/untreated cells. The transfection efficiency in A and B was monitored by cotransfection of a renilla Luciferase construct controlled by a basal promoter. Different experiments having equal results were carried out. B: Titration of 5-FU. Hep3B cells were transiently transfected with the −36/+100 promoter construct. The cells were treated for 48 hours with 1.7 μM etoposide (eto) and increasing concentrations of 5-FU, as indicated. The cells were lyzed and the luciferase activity was determined.

FIG. 5: A region comprising the nucleotides +20 to +100 within 5′UTR of the CD95L gene is responsible for ligand induction following chemotherapy A: Survey of 5′UTR of the CD95L gene. Bars represent the −36/+100 and −36/+100 constructs. The framed sequence is the AP-1 site in the vicinity of the first ATG codon. The arrow shows the translation startsite. B: Hep3B cells were transfected with the constructs described in A, and the luciferase activity was measured following 48-hour treatment with 5-FU (100 μg/ml). A representative experiment of five independent experiments (3-fold samples) is shown. Pnull.luc is a construct used as a negative control without promoter. The transfection efficiency was monitored by means of cotransfection with a renilla-luciferase construct.

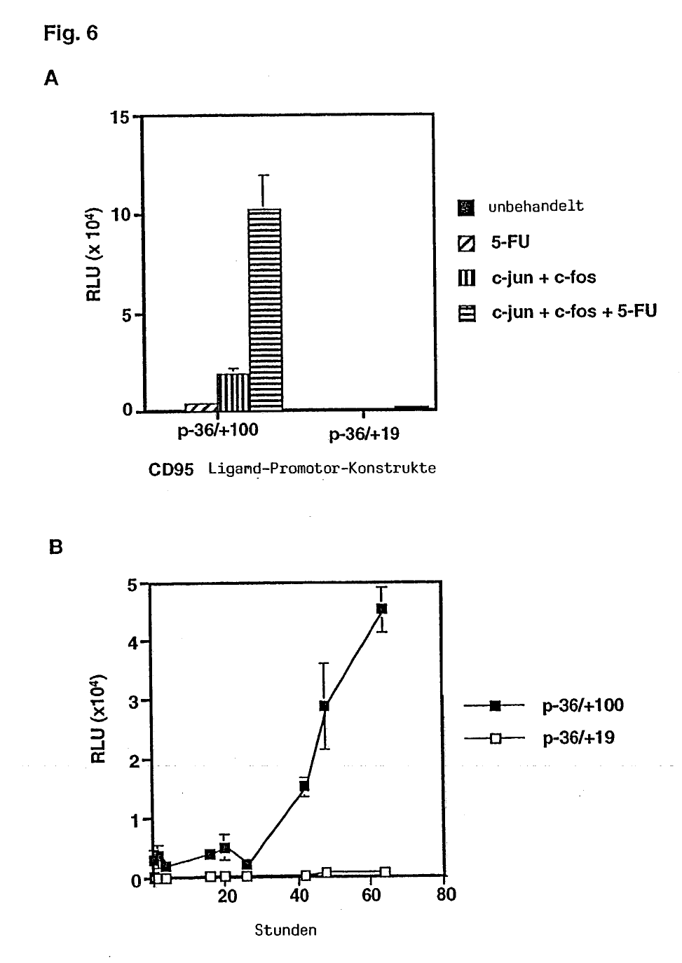

FIG. 6: The −36/+100 and −36/+19 constructs show different behavior as regards the kinetics of activation and inducibility by cotransfection with c-jun and c-fos A: Cotransfection experiments in Hep3B cells with expression vectors for c-jun and c-fos. The cotransfected cells were then treated with 100 μg/ml 5-FU for 48 hours or not treated. The transfection efficiency was normalized by either cotransfection of an expression vector for chloramphenicol transferase (CAT) or renilla luciferase (both controlled by a basal promoter). The luciferase activity was measured using the “dual luciferase” assay (Promega company) in accordance with the manufacturer's instructions, and the CAT activity was determined by means of a commercially available CAT ELISA. Average values (with standard deviation) from different independent experiments are shown. B: “Dual luciferase” assay with Hep3B cells cotransfected with the described CD95L promoter construct and a renilla-luciferase expression vector (as a control for the transfection efficiency). The cells were collected at the indicated times. In addition, the protein content of the transfected cells was determined. The data are average values (with standard deviation) from three samples of a representative experiment. Four independent experiments were carried out.

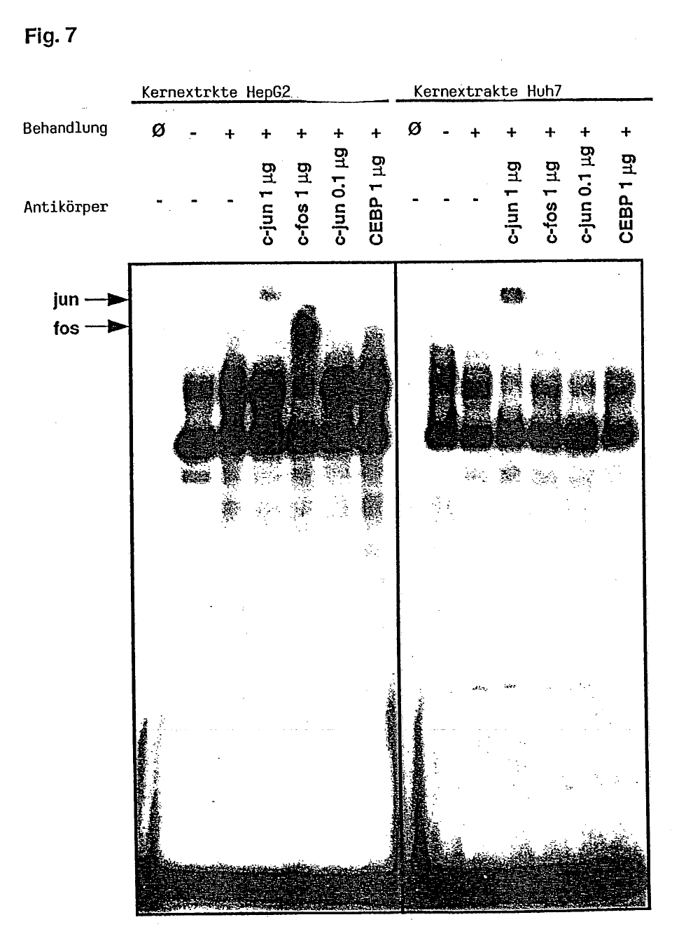

FIG. 7: Nuclear extracts from liver cell lines treated with chemotherapeutic agents shift the mobility of an oligonucleotide containing the AP-1 sequence in the CD95L promoter EMSA and “supershift” analyses of the +73/+99 region of the human CD95L promoter sequence were carried out. Nuclear extracts from HepG2 and Huh7 cells were prepared as described in Example 1. The cells were either treated with 100 μg/ml 5-FU for 48 hours or not treated. EMSA analyses were carried out as described not long ago. Antibodies against c-June or c-Fos were added for the “supershift” analyses. The isotype-adapted anti-C/EBP antibody was used as a control. Ø: negative control without nuclear extracts; −: untreated cells; +: treated cells. Antibodies were added as indicated.

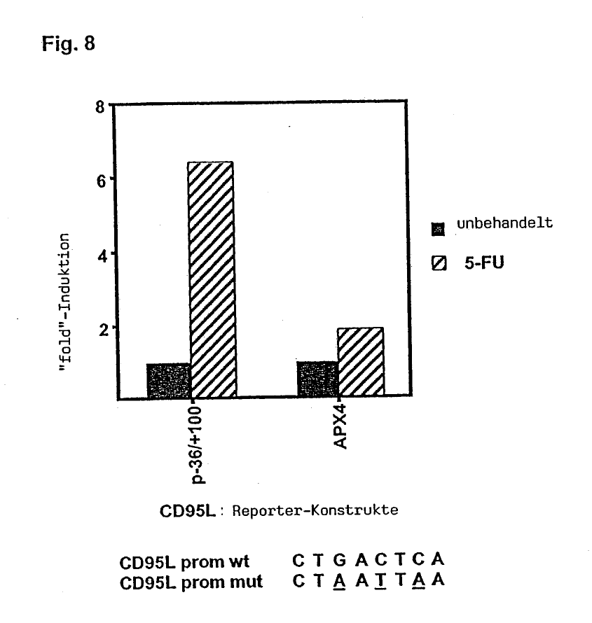

FIG. 8: Mutations within the AP-1 site destroy the inducibility of the promoter construct The AP-1 site in the basal promoter was mutated as indicated. Hep3B cells were transfected with the −36/+100 construct (CD95L prom wt) or the APX4 construct (mutated −36/+100 CD95L.luc; Cd95L prom mut). The transfection efficiency was monitored by cotransfection with renilla luciferase. Following transfection the cells were treated with 5-FU (100 μg/ml) for 48 hours. The luciferase activity was measured and the multiplication of induction was calculated. A representative experiment (of 5 experiments in toto) is shown.

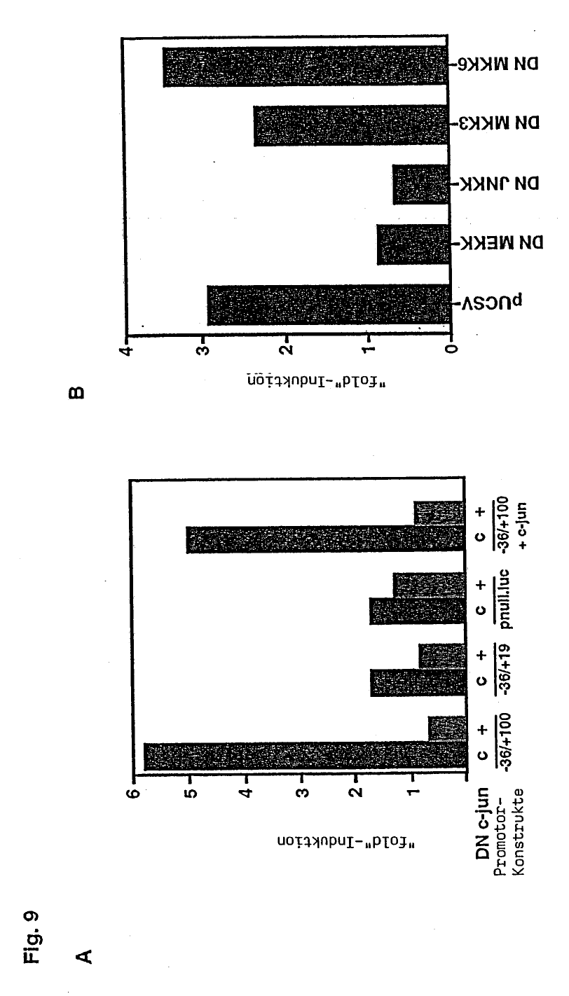

FIG. 9: Dominant-negative c-jun and dominant-negative MEKK-1 and JNKK-1 constructs inhibit the promoter activation following chemotherapy A: Influence of DN c-jun. Hep3B cells were transfected with the −36/+100, −36/+19 or pnull.luc construct. The cells were cotransfected with either a control plasmid (c) or an expression construct for dominant-negative c-jun (+). As a control, c-jun was additionally cotransfected in an experiment. Having concluded the transfection, the cells were divided. Half of them were treated with 100 μg/ml 5-FU, the other half was not treated. The bars show the multiplication of induction which was calculated as follows: RLU (treated cells)/RLU (untreated cells). Relative luciferase units were normalized by means of the renilla-luciferase activity using the “dual luciferase” system. Three independent experiments were carried out one of which is shown. B: Influence of the JNK/SAPK cascade. Cotranfection experiments were carried out with a control vector (pUCSV) or dominant-negative mutants for the stress-activated proteinkinases JNKK-1, MEKK-1, MKK3 and MKK6. The data as regards the multiplication of induction were calculated as in A. One of three independent experiments is shown.

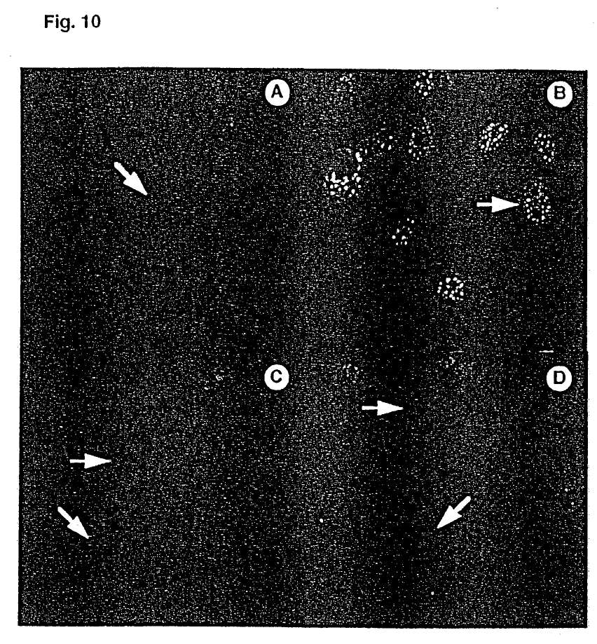

FIG. 10: C-jun is regulated upward in Hep3B cells and human primary hepatocytes following treatment with chemotherapeutic agents A+B: Hep3B cells were seeded onto LabTek™ culture dishes and cultured for two days. Thereafter, the cells were either not treated (A) or treated with 5-FU (100 μg/ml) for 40 hours (B), fixed and stained as described. C+D: Human primary hepatocytes were isolated as described in Example 1 and seeded onto culture dishes. Thereafter, the cells were either not treated (C) or treated with 5-FU (50 μg/ml) for 40 hours (D), fixed and stained with an antibody specific to c-Jun.

The following examples explain the invention.

### EXAMPLE 1

### General Method

(A) Cell Lines

The following cell lines were used: 1) HepG2 derived from human liver blastoma and expressing wild-type p53 with low concentration, 2) Huh7 derived from a human hepatocellular carcinoma and expressing a mutated form of p53 where point mutation to codon 220 results in a shorter half life of p53, 3) Hep3B derived from a human hepatocellular carcinoma and deficient for p53, and 4) SKW6.4, a human T-cell leukemia cell line. HepG2, Hu7 and HcpG3 cells were cultured in DMEM (Gibco BRL, Eggenstein, Germany) which had been supplemented with 10% heat-inactivated fetal calf serum (FCS) (Gibco BRL), 10 mM HEPES (Gibco BRL), 5 mM L-glutamine (Gibco BRL) and 100 μg/ml gentamcyin (Gibco BRL). SKW6.4 cells were stored in RPMI medium (Gibco BRL) containing 10% FCS (Gibco BRL), 10 mM HEPES (Gibco BRL), 2 mM L-glutamine (Gibco BRL) and 100 μg/ml gentamycin (Gibco BRL).

(B) Isolation and Culturing of Primary Human Hepatocytes

Primary human hepatocytes were obtained by means of a 2-step perfusion technique from healthy liver tissue of patients undergoing partial liver resection. For this purpose, a cannula was introduced into a blood vessel of the removed liver tissue and it was perfused with Ca-free and Mg-free “Hank's balanced salt solution (HBSS; Gibco BRL) containing 0.5 mM EDTA and 50 mM HEPES for 15 to 20 min. The perfusion was continued for 15 to 25 min. with “William's medium E” (WME; Gibco BRL) which contained 0.05% type IV collagenase (Sigma) and 5 mM CaCl₂. The cells were separated mechanically from the liver capsule, and the resulting cell suspension was filtered and washed in ice-cold WME until it was free from serum. For separating the hepatocytes from non-parenchymal cells, centrifugation was carried out at 50×g with Percoll™ for 10 min. (adjusted to a density of 1.065 g/ml; Biochrom, Germany). The cells were washed twice with WME and seeded onto maintenance medium to give a density of 1.0−1.5×10⁵ living cells/cm² on collagen-coated culture plates (type I collagen, Serva Biochemicals, Germany). The viability was determined by means of the trypan blue exclusion method. The maintenance medium used was WME supplemented with the following compounds: 5 mM L-glutamine (Flow Laboratory, Germany), 0.6% glucose, 0.02 M HEPES, 50 μg gentamycin (Sigma), 100 μg/ml penicillin (Flow Laboratory), 100 μg/ml streptomycin (Flow Laboratory), 37 μM inosine (Serva), 17.4% DMSO (Merck, Darmstadt, Germany) and 0.14 E/ml insulin (Serva). On days 1 and 2, the maintenance medium was supplemented with 10% FCS (Gibco BRL). The cells were incubated at 37° C. and with 5% CO₂.

(C) Plasmids

The serial deletion constructs of the CD95L promoter were cloned into the pTATA.luc vector (available from T. Wirth, Institut für medizinische Strahlen- und Zellforschung, Würzburg, Germany) or into the pGL2 base vector (Promega, Madison, Wis., U.S.). The deletion constructs of −2269/+100 to −36/+100 were prepared as described not long ago (Li-Weber et al., European Journal of Immunology 28 (1998), 2373). The −36/+19 vector was constructed as follows: The −36/100-CD95L promoter fragment was cleaved with Psp5II and the resulting smaller fragment was again cloned into the pTATA.luc vector. The sequences of all the constructs were checked and confirmed by means of automated dideoxy sequencing (TOPLAB, Munich, Germany).

Dominant-negative MEKK-1 cells (SRα3-ΔMEKK (K432M)) and dominant-negative JNKK cells (SRα3-JNKK (K611R) were provided by M. Karin (Angel et al., Nature 332 (1988), 166), dominant-negative MKK3 cells (MKK3b-(A)) and dominant-negative MKK6 cells (MKK6b-(A)) were supplied by J. Woodgett (Nishina et al., Development 126 (1999), 505) and the control plasmid pUCSV was obtained from P. Angel. The DN-MEKK, DN-JNKK, DN-MKK3, DN-MKK6 and pUCSV constructs are already described (Angel et al., Nature 332 (1988), 166). Dominant-negative c-jun cells (Δaa 1-192) and the empty control vector pCMV were provided by D. Bohmann (Leppa et al., EMBO Journal 17 (1998), 4404). Mutations into the +90 AP-1 site of the −36/+100 construct (APX-4) were introduced by means of the “QuikChange” mutagenesis kit (Stratagene Corp., La Jolla, Calif., U.S.). The following primers (MWG Biotech GmbH, Ebersberg, Germany) were used for the mutagenesis reaction: APX-4/sense: 5′-CCG TTT GCT GGG GCT GGC CTA ATT AAC CAG CTG CCT CTA GAG G-3′; APX-4/antisense; CCT CTA GAG GCA GCT GGT TAA TTA GGC CAG CCC CAG CAA ACG G-3′. As compared to the wild-type sequence of the CD95L promoter the underlined nucleotides represent mutated sites. The mutations were checked and confirmed by automated sequencing (TOPLAB GmbH, Munich, Germany).

(D) Antibodies

Neutralizing anti-CD95L antibodies (NOK-1) and isotype-adapted control antibodies were obtained from Pharmingen (Hamburg, Germany). Antibodies directed against c-jun, c-fos and CEBP for “supershift” analyses were obtained from Santa Cruz Biotechnology Inc. (Heidelberg, Germany). The cells were treated with mAb IgG3-anti-APO-1 at a concentration of 100 ng/ml as described not long ago (Berndt et al., PNAS U.S.A. 95 (1998), 12556).

(E) Treatment with Chemotherapeutic Agents

Cell cultures were treated with bleomycin (cell pharm GmbH, Hanover, Germany) within a dose range of 1 μg/ml to 1 mg/ml, 5-FU (ribosepharm GmbH, Munich, Germany) at a concentration of 10 μg/ml to 150 μg/ml, etoposide (Bristol-Myers Sqibb GmbH, Munich, Germany) within a dose range of 1 to 5 μM, or cisplatin (ribosepharm GmbH, Munich, Germany) within a dose range of 0.5 μg/ml to 2.0 μg/ml. The cells were incubated with the different chemotherapeutic agents for 2 to 64 hours. The clinically relevant concentrations for cancer therapy in humans are as follows: bleomycin: 1.5 μg/ml to 3.0 μg/ml; 5-FU:−; etoposide: −; cisplatin: 0.4 μg/ml to 1.6 μg/ml.

(F) Measurement of Cell Death

Following the corresponding treatment, the cells were trypsinated with 1% trypsin-EDTA for 5 min., washed twice with PBS and stained using 2.5 μg/ml propidium iodide (PI; Sigma). The stain uptake was measured by means of a “FACScan” cytometer (Becton Dickinson GmbH, Heidelberg, Germany) using the “CellQuest” Software. Changes in the forward/side scatter (FSC/SSC) of the cell population were evaluated as well. For quantifying the DNA fragmentation the supernatants were centrifuged at 200×g, the cells were trypsinated and washed as indicated above. Cells were lyzed together with the cell debris in a hypotonic lysis buffer (0.1% sodium citrate and 0.1% triton x100™) containing 50 μg/ml propidium iodide. The incubation was carried out at 4° C. overnight. The nuclei were then analyzed as regards their DNA content by means of flow cytometry (Nicoletti et al., Journal of Immunological Methods 139 (1991), 271).

(G) ⁵¹Cr Release Assay

CD95L-mediated cytotoxicity induced by chemotherapy was studied. Hep3B cells (effector cells) were seeded into 96-well plates and treated with chemotherapeutic agents for 48 hours. After two days, the medium on the effector cells was exchanged. SWK6.4 cells (target cells) were incubated in Na₂⁵¹CrO₄ (100 μCi; NEN, Neu-Isenburg, Germany) for 30 min. Thereafter, labeled cells were added to the effector cells. After 12 to 16 hours, 100 μl supernatant of each well were collected and measured in a gamma counter. The specific lysis was calculated in accordance with formula L=(E−S)/(T−S), E representing the gamma counts of the unknown sample, S being the spontaneous lysis of the labeled target cells in the medium without effector cells, and T representing the maximum release of the target cells kept in 2 N HCl. Further analysis of the assay was only made in case S/T≦30%. Every experiment was carried out three times.

(H) Detection of the CD95L-mRNA expression by means of RT-PCR RNA from different sources was purified by means of the RNeasy” kit (Quiagen GmbH, Hilden, Germany) in accordance with the manufacturer's instructions. 5×10⁶-10⁷ cells of HepG2 and HepB3 cell lines were used for each isolation. 1 μg whole RNA was carried out using MMLV-RT (Gibco BRL) with oligo(dT)₁₅ primers (Roche Diagnostics, Heidelberg, Germany) in a 20 μl reaction, the reaction mixture containing 10 mM DTT and 500 μM dNTPs. 5 μl aliquots were amplified in a DNA thermocycler (Stratagene, Heidelberg, Germany) with 0.5 E Taq DNA polymerase (Roche Diagnostics) in a 50 μl reaction. 35 cycles were carried out (denaturation step: 30 sec. 94° C.; attachment step: 30 sec. 56° C.; extension step: 30 sec. 72° C.). The reaction was concluded by means of an extension step (10 min. 72° C.).

Primers were obtained from MWG Biotech GmbH, Ebersberg, Germany. Their sequences were as follows: CD95L/sense: 5′ATG TTT CAG CTC TTC CAC CTA CAG A-3′; CD95L/antisense: 5′-CCA GAG AGA GCT CAG ATA CGT TGA C-3′, a PCR product having a length of 500 bp being obtained. All the primers cover three introns of CD95L, thus facilitating a differentiation between cDNA and genomic DNA. Every reversely transcribed mRNA was checked as an internal control by means of β-actin-PCR, the following PCR primers being used: β-actin/sense: 5′TGA CGG GGT CAC CCA CAC TGT GCC CAT CTA-3′; and β-actin/antisense: 5′-CTA GAA TTT GCG GTG GAC GAT GGA GGG-3′, a PCR product having a length of 600 bp being obtained. PCR products were analyzed on 1.5-2% TBE (Tris-borate-EDTA) agarose gels.

(I) Transient Transfections

One day before the transfection, the cells were plated out with a density of 0.6×10⁶/9 cm Petri dishes. The medium was exchanged two hours before the transfection. The transfection was carried out by means of the method based on calcium phosphate precipitation. In summary, 5 to 40 μg DNA were diluted in 500 μl of a calcium chloride solution. The DNA/calcium chloride mixture was added drop-wise to 500 μl HBS. Precipitation was carried out at room temperature for 20 min., and then the precipitation mixture was added to the culture medium. The precipitate was allowed to stay on the cells for 12 to 24 hours. Thereafter, the cells were shock-treated using 16% glycerin in full medium for 3 min. Following three wash steps using PBS, the cells were cultured in medium supplemented with 20% FCS for at least six hours. Finally, the cells were distributed over 6-well plates, and the chemotherapeutic treatment was started for different periods.

(J) Luciferase Assay

After three wash steps in PBS, the cells were lyzed in 200 μl passive lysis buffer (Promega). Following 20 minutes of incubation at room temperature, the cells were scratched off the plates. The lyzates were subjected to two freeze/thaw cycles and then cell debris was removed by centrifugation. The supernatants were measured in a “duolumat” device (Berthold company, Wildbach, Germany) by means of the “dual luciferase” assay system from Promega. To normalize the transfection efficiencies, a renilla-luciferase expression vector, pRenilla, controlled by a basal promoter was used. Renilla luciferase was measured in the “dual luciferase” assay system, and CAT expression was determined by means of a commercially available ELISA (Roche Diagnostics). In addition, the protein amount was measured in the protein assay from Biorad (Munich, Germany) and used to normalize the protein content of the transfected cells.

(K) EMSA and “Supershift” Analyses

Nuclear extracts from HepG2 and Huh7 cells were prepared. In summary, 4×10⁷ cells were lyzed in 10 mM Tris-HCl, pH 7.4, 2 mM MgCl₂, 140 mM NaCl, 0.5 mM DTT, 0.5 mM PMSF and 0.1% triton-X100. Thereafter, sucrose density gradient centrifugation was carried out, and the nuclear fraction was separated in 20 mM HEPES, pH 7.9, 25% glycerin, 0.42 M NaCl, 1.5 MgCl₂, 0.2 mM EDTA, 0.5 mM DTT and 0.5 mM PMSF. Following 30 minutes of centrifugation, the nuclear membranes were pelleted, and the supernatants were stored in liquid nitrogen after determining the protein content with the above described assay.

The double-stranded oligonucleotides containing the AP-1 site at +90 in the CD95L promoter were end-labeled by means of T4 polynucleotide kinase (MBI Fermentas, St.Leon-Roth, Germany) with [γ-³²P]ATP (5000 Ci/mmol; Amersham GmbH, Braunschweig, Germany). The single-stranded oligonucleotides had the following sequences: sense 5′-GGG CTG GCC TGA CTC ACC AGC TGC-3′; and antisense: 5′-GCA GCT GGT GAG TCA GGC CAG CCC-3′. Free nucleotides were removed using “microspin G-50” columns (Pharmacia GmbH, Freiburg, Germany).

The binding reactions were carried out at 4° C. for 30 min. using 5 μg nuclear protein in a buffer containing 100 ng/μl BSA (Roche Diagnostics), 50 ng/μl poly[d(I-C)] (Roche Diagnostics], 2 mM DTT (Gibco BRL), 500 μM “Pefablock” (Roche Diagnostics), 1 μg/ml aprotinine (Roche Diagnostics), 25 mM HEPES, 5 mM MgCl₂ (Sigma), 35 mM KCl (Sigma) and 3×10⁴ cpm of the labeled oligonculeotide. For the supershift analyses, 1 μg antibodies were added to the binding reaction. The samples were separated on a non-denaturing 6% polyacrylamide gel in 0.5% TBE, and an autoradiography was made overnight.

(L) Immunofluorescence Studies

Cultured Hep3B cells or freshly isolated human primary hepatocytes were plated onto Lab-Tek™ chamber slides (Renner GmbH, Darmstadt, Germany). Following culturing over a period of at least two days, the cells were treated with chemotherapeutic agents. Thereafter, fixation in methanol/acetone (5 min. or 10 sec. at −20° C.) was carried out. Then, the cells were initially incubated at 37° C. for 1 hour with the specific primary antibody against human c-Jun (Santa Cruz GmbH, Heidelberg, Germany), and after three wash steps using PBS, the cells were covered with fluorescein-isothiocyanate-conjugated goat anti-rabbit IgG (Dianova, Hamburg, Germany) for 1 hour. Non-specific staining was monitored by incubation with mouse or rabbit immunoglobulins in place of the specific primary antibody or by blocking the primary antibody with the immunogenic peptide. The slides were covered with cover glasses and evaluated by means of a fluorescence microscope.

### EXAMPLE 2:

### Chemotherapeutic Agent-Induced Apoptosis in Liver Cell Cancer Cell Lines Via the CD95/CD95L System

HepG2 cell cultures were treated at 70% confluence with 5-FU in either the absence or presence of a CD95L-blocking compound, and apoptosis was determined by PI exclusion and analysis of the forward/side scatter (FSC/SSC). As evident from FIG. 1, the chemotherapeutic treatment results in a significant increase in the apoptosis rate, this event occurring 12 to 24 hours following the administration of 5-FU and reaching 43% after 48 hours. These results were confirmed by staining the cells as regards the subdiploid DNA content. The effect of the chemotherapeutic agents could substantially be blocked by the simultaneous administration of 50 μg/ml CD95-Fc or 50 μg/ml NOK-1 antibodies, which indicates a major influence of the CD95 system on chemotherapy-induced apoptosis. Similar results were obtained by treatment with etoposide, an inhibiting agent of topoisomerases.

### EXAMPLE 3:

### CD95L is Regulated Upward Following the Administration of Chemotherapeutic Agents

Since it could be shown that CD95/CD95L interactions are of significance for inducing apoptosis after the influence of chemotherapeutic agents, the potential upward regulation of CD95L after the stimulation was studied. As evident from FIG. 2A, CD95L-mRNA is regulated upward after treatment with bleomycin, a chemotherapeutic agent belonging to the group of antibiotics. All of the concentrations used were within the clinically relevant range. The upward regulation was observable in both HepG2 (wt p53) and Hep3B (p53 −/−) cells, and is thus independent of p53, since the Hep3B cell line lacks this protein.

5-FU and etoposide can regulate upward CD95L in both HepG2 and Hep3B cells as a function of time and dose (FIGS. 2B and 2C). The upward regulation of CD95L is delayed and occurs in temporal relation with respect to the occurrence of apoptosis in liver cell cancer cell lines after 20 to 25 hours. In order to elucidate the mechanism underlying the upward regulation of CD95L in the cell lines of liver cell cancer, blocking experiments were carried out with either the transcription inhibitor actinomycin C1, or the translation inhibitor, cycloheximide. Both active substances were used with subtoxic concentrations and they showed no effect on the transcription or translation of the “housekeeping” gene β-actin. The upward regulation of CD95L following treatment with chemotherapeutic agents was, however, reduced efficiently (FIGS. 2D and 2E), which indicates a regulation of CD95L on the transcription level.

In order to study whether the upward regulation on the mRNA level coincides with an upward regulation of the CD95L protein on the cell surface, the capacity of treated Hep3B cells of killing CD95-positive target cells was studied. ⁵¹Cr-labeled SKW6.4 cells, a B cell line expressing high CD95 levels, were incubated on a Hep3B cell monolayer overnight. The Hep3B cells were either not treated or treated with 100 μg/ml 5-FU 48 hours before their incubation with SKW6.4 cells. After collecting the supernatant, the released ⁵¹Cr activity was measured. As shown in FIG. 3, the treatment of Hep3B cells with 5-FU results in a significant killing of CD95-bearing SKW6.4 cells, which indicates an interaction between CD95 and CD95L. In order to further study this effect, blocking experiments were carried out with the NOK-1 antibody. As shown in FIG. 3, the inhibition of CD95L almost fully inhibits cell death, only a residual killing activity of about 5% being observable. In summary, these data prove that chemotherapeutic agents result in an upward regulation of functional CD95L protein on the cell surface.

### EXAMPLE 4

### Chemotherapeutic Agents Exert Their Influence on the CD95L Promoter, Synergistic Effects Being Observable

Since due to the upward regulation observed on the transcription level it was assumed that chemotherapeutic agents exert their influence directly on the CD95L promoter, different luciferase reporter constructs were prepared, each controlled by a certain fragment from the 5′-non-translated region of the CD95L gene. The −1204/+100 and −860/+100, −345/+100 and −36/+100 constructs have already been described and can well be induced in jurkat T cell lines in transient transfection assays following stimulation with phorbol ester and ionomycon (Li-Weber et al., European Journal of Immunology 28 (1998), 2373). In order to study in more detail the effect of chemotherapeutic agents on liver cells, Hep3B cells were transfected transiently with the different constructs, and the cells were then stimulated by treatment with 5-FU, etoposide or a combination of both compounds. All of the above-described constructs were likewise inducible. A representative experiment for the −36/+100 construct is shown in FIG. 4. The combination of different chemotherapeutic agents shows a synergistic effect on all of the constructs (FIG. 4A). In addition, these active substances show great dose-dependency (FIG. 4B), which indicates that the promoter regulation takes place at physiological concentrations of the chemotherapeutic agents, since the experiments were carried out in a clinically relevant dose range.

### EXAMPLE 5

### CD95L is Regulated Upward in Liver Cells Following Stimulation with Chemotherapeutic Agents by Activating the Newly Identified AP-1 Element.

Since all of the promoter constructs ranging from −1204/+100 to −36/+100 showed equal inducibility following stimulation with chemotherapeutic agents, it was assumed that a promoter element located between −36 and the translation start might be responsible for promoter activation. In order to further restrict this assumed site, 3′-deletion constructs of the CD95L promoter were produced. One of these constructs (−36/+19) was used for further experiments, and this is shown by way of diagram in FIG. 5A as compared to the −36/+100 luciferase construct. As shown in FIG. 5B, this construct showed a significantly reduced basal activity and was also less inducible than the −36/+100 construct. Hence it was concluded that the +20/+100 range of the CD95L promoter contains an element which is responsible for the inducibility by stimulation with chemotherapeutic agents.

A computer search for binding sites for transcription factors yielded a consensus sequence for the dimeric factor AP-1. The accurate position of this sequence is shown in FIG. 5A (framed). In order to confirm that this site is actually a functional AP-1 site, c-jun and c-fos constructs were transfected together with the −36/+100 and −36/+19 luciferase reporter constructs. As shown in FIG. 6A, cotransfection with c-jun and c-fos revealed a clear stimulation of the −36/+100 construct containing the AP-1 site but not that of the −36/+19 construct which lacks this site at +90.

Transfection with c-jun or c-fos alone also led to a stimulating effect. Cotransfection with the AP-1 components and treatment with 5-FU did not result in a significant activity increase of the −36/+19 construct, which clearly refers to the lack of the responsible promoter fragment. On the contrary, treatment of cotransfected cells with 5-FU resulted in another increase in the luciferase activity in the −36/+100 construct, comparable to induction without transfection with c-jun and c-fos.

For further studying the significance of the AP-1 site for the induction by chemotherapeutic agents, kinetic studies were carried out (FIG. 6B). The −36/+100 construct reacted with a major activation which started 20 to 25 hours following the initiation of the 5-FU treatment. The activation then proceeded and reached its maximum level after 64 hours. The −36/+19 construct, however, did not respond to this treatment. Even after incubation with 5-FU extended to 64 hours no significant increase was observable. These results also emphasize the significance of the AP-1 site for the chemotherapeutic treatment.

Since the AP-1 complex can be formed by various constituents effecting the response to a certain cellular stimulus, studies as to the structure of the dimer bond to the promoter were conducted. For this purpose, “gelshift” analyses were carried out with oligonucleotides comprising the consensus AP-1 site (CCTGACTC) in the CD95L promoter. It was possible to shift these oligonucleotides by nuclear extracts from 5-FU-treated HepG2 cells and 5-FU-treated Huh7 cells (another liver cell carcinoma cell line) as regards their mobility, only a minor mobility shift being observable with extracts from untreated cells (FIG. 7). It was possible to eliminate competitively this complex formation with unlabeled wild-type oligonucleotide and with a consensus AP-1-oligonucleotide, but not with unlabeled oligonucleotides which contained the consensus sequences for either NF-κB or SP-1, which clearly indicates a specific binding of AP-1 to the target site within the promoter. In order to identify partial components of the binding complex, “supershift” experiments were conducted. Antibodies against c-Jun and c-Fos further shifted the mobility of the complex (FIG. 7). Anti-c-Jun and anti-c-Fos antibodies shifted the mobility of the complexes with nuclear extracts from both cell types (hepG2 and Huh7), however, to a different extent. Anti-c-Fos antibodies led preferably in nuclear HepG2 extracts to a mobility shift and anti-c-Jun antibodies did this preferably in Huh7 extracts. The mobility of the complexes was not influenced by either antibodies against C/EBP (as an isotype-adapted control) (FIG. 7) or antibodies against JunD or ATF2, which indicates a preference of the Jun/Fos heterodimer for this site.

As another detection regarding the significance of this CD95L promoter site for the upward regulation resulting from the chemotherapeutic treatment, this AP-1 element was mutated by site-directed mutagenesis. The consensus wild-type sequence CTGACTCA was mutated at three different positions with respect to CTAATTAA. These mutations destroy the willingness to respond of an AP-1 luciferase reporter construct. The introduction of these mutations into the −36/+100 reporter construct almost fully eliminated the inducibility of the construct after 48-hour treatment with 100 μg/ml 5-FU (FIG. 8). This is another evidence for the significance of the AP-1 site in the case of chemotherapy-induced apoptosis.

### EXAMPLE 6

### Involvement of the Stress-Activated Protein Kinase

Pathway (SAPK/JNK) in the Chemotherapy-Induced Upward Regulation of CD95L

In many cell stress-triggering factors, SAPK/JNK cascade is involved in the target cells. Chemotherapeutic agents represent major stress factors in both in vivo and in vitro. Therefore, it was studied whether this system of successively activated kinases is involved in the response to chemotherapeutic agents. The last objective of the SAPK cascade is the posphorylation of c-Jun. Therefore, co-transfections of a dominant-negative c-jun construct (DN c-jun) which lacked amino acids 1 to 192 (a region comprising the phosphorylation sites at the serine residues 63 and 73) were carried out with the different luciferase reporter constructs. Dominant-negative c-jun considerably impairs the activation of the AP-1 complex, since although it still contains the dimerization domain, it can no longer activate the AP-1 target genes. The effectiveness of inhibition is shown in FIG. 9A. Cotransfection of Hep3B cells with the −36/+100 reporter construct, c-jun and dominant-negative c-jun fully eliminated the activating effect of c-jun on the promoter (FIG. 9A, two columns on the right). However, it is much more relevant that dominant-negative c-jun drastically inhibits the promoter activation after treatment with chemotherapeutic agents, which emphasizes the necessity of the c-jun phosphorylation for the observed upward regulation (FIG. 9A).

The involvement of stress-activated kinases in the activation of the CD95L promoter was finally studied. For this purpose, Hep3B cells were cotransfected with dominant-negative constructs of different SAPK/JNKs. As shown in FIG. 9B, it was possible to inhibit the activation by 5-FU by cotransfection of dominant-negative MEKK-1 and dominant-negative JNKK, which indicates the successive activation of the MEKK-1 [symbol] JNKK [symbol] JNK [symbol] c-Jun axis. As a control, a dominant-negative mutant of the p38 pathway was used. Both dominant-negative constructs of the p38 pathway (DN-MKK3 and DN-MKK6) had virtually no influence on the upward regulation of promoter activation following treatment with cytostatic agents. Thus, it can be assumed that chemotherapeutic agents induced CD95L via the above-mentioned JNK pathway.

Since AP-1 represents a protein of the acute phase and is usually activated within some minutes following a given stimulus, it was studied whether an increased activation of AP-1 can be observed within a certain period fitting in the upward regulation of CD95L found in the above experimental system. For this purpose, Hep3B cells were immunostained with an antibody against c-Jun 40 hours following treatment with 5-FU. After the immunofluorescence staining, the intensity of the color and the spatial distribution of positive signals were studied. The specificity of the color was confirmed by blocking experiments using antigenic peptides. Untreated Hep3B cells showed slight positive signals (FIG. 10A). After treatment with 5-FU for 40 hours, the cells widely accumulated AP-1 within the nucleus, which can be seen as dot-like positive signals in FIG. 10B. This finding indicates that following extensive treatment with chemotherapeutic agents, major activation of AP-1 occurs. It was also studied whether this phenomenon occurs in primary human cells as well. For this purpose, primary human hepatocytes obtained from patient material during liver transplantation were isolated. The isolated cells were cultured for two days to eliminate stress effects on account of the isolation methods and then treated with 5-FU for 40 hours, fixed and immunostained. The cultured primary human hepatocytes were negative as regards AP-1 (FIG. 10C). Following 40 hours of chemotherapeutic treatment, the primary hepatocytes showed an intensive nuclear reactivity spread over the entire nucleus (FIG. 10D), indicating that in the primary human hepatocytes a reaction type proceeds the same as that in the cell lines of liver cell cancer.

1. A method of studying whether a test compound is effective for a chemotherapy, wherein the method comprises the steps of:
a) providing a DNA sequence which comprises the AP-1 site shown in FIG. 5A as a constituent of a promoter functionally linked with a reporter gene or CD95L gene;
b) contacting the DNA sequence from a) with the test compound in a cellular assay; and
c) determining the activation of the promoter, said activation indicating that the test compound is effective for a chemotherapy.

2. The method according to claim 1, wherein the promoter is the CD95L promoter.

3. The method according to claim 1 or 2, wherein the test compound forms part of a substance library.

4. The method according to any of claims 1 to 3, wherein the DNA sequence comprising the AP-1 site is the nucleic acid sequence from positions −36 to +100, shown in FIG. 5A, or a fragment thereof.

5. The method according to claim 4, wherein the DNA sequence comprising the AP-1 site is the nucleic acid sequence from positions +84 to +91, shown in FIG. 5A.

6. The method according to any one of claims 1 to 5, wherein the reporter gene is the CAT, luciferase, LacZ or GFP gene.

7. A medicament which comprises a mixture of at least two compounds identified according to the method as defined in any of claims 1 to 6 and/or supporting the expression of the CD95L gene.

8. Use of the mixture defined in claim 7 for treating cancer and/or autoimmune diseases.


SEQUENCE LISTING (9 sequences)

SEQ ID NO: 1 - DNA, 43 bp, Artificial Sequence
  Synthetic Construct
ccgtttgctggggctggcctaattaaccagctgcctctagagg

SEQ ID NO: 2 - DNA, 43 bp, Artificial Sequence
  Synthetic Construct
cctctagaggcagctggttaattaggccagccccagcaaacgg

SEQ ID NO: 3 - DNA, 25 bp, Artificial Sequence
  Synthetic Construct
atgtttcagctcttccacctacaga

SEQ ID NO: 4 - DNA, 25 bp, Artificial Sequence
  Synthetic Construct
ccagagagagctcagatacgttgac

SEQ ID NO: 5 - DNA, 30 bp, Artificial Sequence
  Synthetic Construct
tgacggggtcacccacactgtgcccatcta

SEQ ID NO: 6 - DNA, 27 bp, Artificial Sequence
  Synthetic Construct
ctagaatttgcggtggacgatggaggg

SEQ ID NO: 7 - DNA, 24 bp, Artificial Sequence
  Synthetic Construct
gggctggcctgactcaccagctgc

SEQ ID NO: 8 - DNA, 24 bp, Artificial Sequence
  Synthetic Construct
gcagctggtgagtcaggccagccc

SEQ ID NO: 9 - DNA, 52 bp, Artificial Sequence
  Synthetic Construct
gagaagaagtaaaaccgtttgctggggctggcctgactcaccagctgccatg